(19) United States
(12) Patent Application Publication
(10) Pub. No.: US 2010/0215716 A1
(43) Pub. Date: Aug. 26, 2010

(54) COMPOSITIONS AND METHODS FOR COATING ORTHOPEDIC IMPLANTS

(75) Inventors: Karen S. TROXEL, Warsaw, IN (US); Sona SUNDARAMURTHY, Warsaw, IN (US); Ellizabeth PEREPEZKO, Warsaw, IN (US)

Correspondence Address: HARNESS, DICKEY & PIERCE, P.L.C., P.O. BOX 828, BLOOMFIELD HILLS, MI, 48303, US

(73) Assignee: Biomet Manufacturing Corp., Warsaw, IN (US)

(21) Appl. No.: 12/710,859

(22) Filed: Feb. 23, 2010

Related U.S. Application Data
(60) Provisional application No. 61/154,515, filed on Feb. 23, 2009.

(51) Int. Cl. A61F 2/00; A61F 2/28; A61B 17/56; A61K 31/65; A61K 31/43; A61K 31/545; A61K 31/4025; A61K 31/7048; A61K 31/5383; A61K 38/14; B05D 3/02

(52) U.S. Cl. 424/423; 623/16.11; 606/60; 514/154; 514/192; 514/198; 514/206; 514/422; 514/29; 514/230.2; 514/8; 427/2.26

(57) ABSTRACT

An orthopedic implant suitable for insertion into the body of a subject, the implant comprising: a metal substrate having one or more surfaces operable to contact a bone tissue or soft tissue when implanted into the subject; a coating comprising a resorbable polymer impregnated with an admixture of a rifamycin antibiotic and a second antibiotic selected from the group consisting of tetracyclines, penicillin, ampicillin, cefazolin, clindamycin, erythromycin, levofloxacin, or vancomycin. The adhered coating layer present on the one or more surfaces is capable of releasing the rifamycin and second antibiotics in an antimicrobially effective amount. Method for making an antibiotic coated implant by mixing a resorbable polymer mixture with an antibiotic solution forming a coating solution, applying the coating solution to a surface of the implant and evaporating the solvent from the coated layer.

### CROSS-REFERENCE TO RELATED APPLICATION

This application claims the benefit of U.S. Provisional Application No. 61/154,515 filed Feb. 23, 2009. The entire disclosure of the above application is incorporated herein by reference.

### BACKGROUND

The present technology relates to resorbable antibiotic coatings for orthopedic implants, methods for making the same and orthopedic implants produced therefrom.

Implantable orthopedic devices are commonly implanted into the body of a human or animal subject or patient for orthopedic purposes such as to strengthen bones, fasten portions of a bone to correct a fracture, and to replace joints such as knees, hips and elbows. Orthopedic fixation devices are used in the treatment of fractures, soft-tissue injuries, and reconstructive surgery. After fracture reduction, internal, external, or intramedullary fixation devices may be used to provide stability and maintain the alignment of bone fragments during the healing process. They must be strong and secure enough to allow early mobilization of the injured part, as well as the entire patient. Compression is used whenever possible to increase the contact area and the stability between fragments and to decrease the stress on the implant. Screws are used primarily to provide interfragmental compression or to attach plates, which can then provide compression, prevent displacement, and support the fragments during healing. Pins and wires can be used for fixation of small fragments or fractures in small bones and for attachment of external fixation devices and traction. Such implants are manufactured most commonly with metal.

The outer exposed surfaces of orthopedic devices implanted into the body come into contact with body tissue and fluids. Since they are foreign in nature to the body, they pose a site for growth of bacteria and potential infection. Prevention of infection and sepsis can be achieved by providing orthopedic devices with antimicrobial properties to combat the colonization of bacteria or inhibit their growth. For example, U.S. Pat. No. 5,756,145, Darouiche, issued May 26, 1998 describes the infections that may be caused by medical implants, such as hip joint replacement, and seeks to solve the problem by providing the implant with an antimicrobial coating layer that is covered by one or more protective coating layers. The antimicrobial materials considered are basically of a liquid organic type that requires a protective coating layer. This increases the complexity of providing the implant with the desired antimicrobial property. Furthermore, adding protective coating layers may ultimately prevent the mode of action and elution of the antimicrobials once implanted.

Due to the rapid growth rate and presence of virulence factors, bacteria are able to set up infections within days of the surgical procedure causing loss of implant fixation, local tissue inflammation, and local tissue necrosis due to sepsis. The most common organisms causing these infectious complications are Staphylococcus epidermidis, Staphylococcus aureus and Pseudomonas sp. when the injuries are related to trauma or battlefield injuries. In the case of orthopedic procedures, Staphylococcus epidermidis, Staphylococcus aureus account for almost 70-80% of all infectious organisms, with Staphylococcus epidermidis being the most common organism.

A considerable amount of attention and study has been directed toward preventing such colonization by the use of antimicrobial agents, such as antibiotics and other antimicrobials, bound to the surface of the materials employed in such devices. In such attempts, the objective has been to produce devices coated with bacteriostatic or bactericidal agents to prevent colonization, often employing caustic, carcinogenic and toxic solvents to ensure that the bacteriostatic or bactericidal agents bind to the surfaces of the implantable devices.

There is still a need for orthopedic devices coated with a resorbable antibiotic material that can withstand the rigor of orthopedic surgery, be capable of manufacture without the use of harsh and toxic chemicals, and yet provide prophylactic amounts of antibiotics that are targeted against orthopedic related infections.

### SUMMARY

The present technology provides an orthopedic implant suitable for insertion into the body of a subject, comprising a metal substrate, and a coating comprising a resorbable polymer and an antibiotic. In various embodiments, the antibiotic is an admixture comprising a rifamycin antibiotic and a second antibiotic. The second antibiotic is selected from the group consisting of tetracyclines, penicillin, ampicillin, cefazolin, clindamycin, erythromycin, levofloxacin, vancomycin, and mixtures thereof. For example, the antibiotic can be a mixture of tetracycline and rifamycin antibiotic. The resorbable polymers preferably comprise lactones, such as butyrolactone, valerolactone, caprolactone, propiolactone; dioxanones; glycolide; lactide and mixtures thereof. The coating present on the one or more surfaces is capable of releasing the antibiotics in an antimicrobially effective amount.

The present technology also provides methods of making an orthopedic implant comprising a metal device with one or more surfaces coated with an antibiotic containing resorbable polymer matrix. Such methods comprise a) dissolving a resorbable polymer in a suitable organic polymer solvent forming a resorbable polymer mixture; b) adding rifampin and a second antibiotic selected from the group consisting of tetracyclines, penicillin, ampicillin, cefazolin, clindamycin, erythromycin, levofloxacin, vancomycin, and mixtures thereof to an organic antibiotic solvent forming an antibiotic solution; c) mixing the resorbable polymer mixture with the antibiotic solution forming a coating solution; d) applying the coating solution to the one or more surfaces of the metal implant; and e) evaporating the organic solvent to form an antibiotic containing resorbable polymer matrix coated orthopedic implant. In various embodiments, the resorbable polymer mixture is poly(D,L-lactide-co-caprolactone dissolved in an organic solvent, for example, acetonitrile.

In another aspect, the present technology provides for a method for making or forming an orthopedic implant, the method comprising: a) dissolving a resorbable polymer in a suitable organic polymer solvent forming a resorbable polymer mixture; b) adding a second selected antibiotic and rifampin to an organic antibiotic solvent forming an antibiotic solution; c) mixing the resorbable polymer mixture with the antibiotic solution forming a coating solution; d) applying the coating solution to the one or more surfaces of the metal implant forming an adhesive coating layer; and e) evaporating the organic solvent from the adhesive coating layer to form an antibiotic containing resorbable polymer matrix coated orthopedic implant.

The articles thus produced by the methods disclosed herein include hip implants, knee implants, elbow implants, prosthetic frames, bone prostheses, small joint prostheses, and fixation devices. Internal and external fixation implants and devices include bone plates, bone screws, anchors, intramedullary nails, arthrodesis nails, rods, pins, wires, spacers, and cages. Preferably, the surface or surfaces to be coated with the subject antibiotic resorbable polymer have a surface or surfaces textured uniformly with surface irregularities, including pores (micropores), dimples, spikes, ridges, grooves (e.g., microgrooves), roughened texture (e.g., microtextured), surface grain, strips, ribs, channels, ruts. The size of the micropores, dimples, spikes, ridges, grooves (e.g., microgrooves), roughened texture (e.g., microtextured), surface grain, strips, ribs, channels, ruts can range from about 1 μm to about 500 μm.

### DRAWINGS

FIG. 1 is a graph depicting the elution of minocycline and rifampin from a 5% poly(D,L-lactide-co-caprolactone polymer coated cylinder versus time.

FIG. 2, panels A-D depict photographs of antibiotic containing resorbable polymer coating on the intramedullary femoral nails before and after implantation into a cadaver. Panels A and B show the intramedullary femoral nail coated with the antibiotic containing resorbable polymer. Panels C and D show the same intramedullary femoral nail after being removed from the implantation site. The antibiotic containing resorbable polymer coating is colored differently than the color of the intramedullary femoral nail, thus indicating regions of exfoliation of the coating.

FIG. 3 is a table listing the toxicity of various organic solvents as judged by oral lethal dose evaluated in rats.

FIG. 4 is a table listing flammability data of various organic solvents.

FIG. 5 is a table listing the solubility of various antibiotics and polylactide-co-caprolactone 70:30 in various organic solvents where solubility is defined as soluble as concentrations of at least 0.05 mg of antibiotic per ml of solvent or at least 5% w/v of polymer in organic solvent.

It should be noted that the figures set forth herein are intended to exemplify the general characteristics of materials and methods among those of the present technology, for the purpose of the description of certain embodiments. These figures may not precisely reflect the characteristics of any given embodiment, and are not necessarily intended to define or limit specific embodiments within the scope of this technology.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. A non-limiting discussion of terms and phrases intended to aid understanding of the present technology is provided at the end of this Detailed Description.

The present technology provides orthopedic implants coated at least partially with a resorbable antimicrobial coating. The resorbable antibiotic coating preferably has two or more antibiotics that confer broad spectrum antibacterial activity against bacteria and yeast and other fungal organisms. Preferably, the antimicrobial coating comprises a combination of rifamycin antibiotic and a second antibiotic selected from the group consisting of tetracyclines, penicillin, ampicillin, cefazolin, clindamycin, erythromycin, levofloxacin, or vancomycin, for example, the antibiotics minocycline and rifampin. The antibiotics are impregnated into a resorbable polymer material that when coated on a surface of an orthopedic device, degrades in situ when implanted into a subject. In various embodiments, the orthopedic implant has an adhesive, abrasion resistant thin-layer coating of the resorbable polymer impregnated with the antibiotics rifamycin and a second selected antibiotic.

The orthopedic implants of the present technology include any implant that is at least partially implanted into the body of a subject. As used herein, “at least partially implanted” refers to orthopedic implants that are completely implanted within the body, and those that are partially implanted into the body. The orthopedic implant can include those implants that span across the skin layers interfacing with an internal tissue, such as a hard tissue like bone, or a soft tissue like muscle or cartilage, or with another implant. Orthopedic implants useful in the present technology can also include prosthesis parts and accessory components interfacing such prosthesis parts. Generally, the surfaces of the implant are completely or partially implanted into the body of the subject, comprising a metal substrate having one or more surfaces operable to contact a bone tissue or soft tissue when implanted. Orthopedic implants useful in the present technology may be permanent tissue replacement devices, permanent stabilization devices, or temporary skeletal stabilization devices.

The orthopedic implants of the present technology include prosthetic implants or parts thereof, for example, hip implants, knee implants, elbow implants; prosthetic frames; bone prostheses; small joint prostheses; and fixation devices. Internal and external fixation implants and devices include bone plates, anchors, bone screws, rods, intramedullary nails, arthrodesis nails, pins, wires, spacers, and cages. Such devices are commercially available from leading orthopedic device manufacturers including; Biomet Inc. (Warsaw, Ind., USA). Other manufacturers can include Zimmer, Inc. (Warsaw, Ind., USA) and DePuy Orthopedics, Inc. (Warsaw Ind., USA) and DePuy Spine, Inc. (Raynham, Mass., USA).

The orthopedic implants of the present technology can comprise solid metals, for example gold, silver, stainless steel, platinum, palladium, iridium, iron, nickel, copper, titanium, aluminum, chromium, cobalt, molybdenum, vanadium, tantalum, and alloys thereof. In preferred embodiments, the orthopedic implant comprises a metal including surgical stainless steel, titanium or a titanium alloy.

One or more surfaces of the metal substrate, for example the surface being coated with the resorbable antimicrobial coating, may be textured. The orthopedic implant surface coated with a resorbable antimicrobial coating can be textured uniformly with surface irregularities, including pores (micropores), dimples, spikes, ridges, grooves (e.g., microgrooves), roughened texture (e.g., microtextured), surface grain, strips, ribs, channels, ruts. The size of the micropores, dimples, spikes, ridges, grooves (e.g., microgrooves), roughened texture (e.g., microtextured), surface grain, strips, ribs, channels, ruts can range from about 1 μm to about 500 μm. In some embodiments, the size ranges from about 10 μm to about 100 μm. The texture may be formed by any suitable methods, for example, by molding, chemical etching, roughening with sandpaper or other abrasives (e.g., sand blasting and glass bead blasting), electrical means (such as EDM machining), thermal means, or laser etching.

In accordance with the present technology, the orthopedic implants described above can be coated with a resorbable antibiotic coating on at least one surface of the orthopedic implant. In some embodiments, all surfaces of the implant exposed to body tissues are coated. Preferably, in other embodiments, the surfaces of the orthopedic implant to be coated with the antibiotic coatings are surfaces that are not intended to provide a structural network for tissue or cellular ingrowth.

The resorbable antibiotic coating may contain one or more resorbable polymers that are degraded in vivo over time. As used herein, the term “resorbable” includes within the penumbra of its meaning, bioresorbable, biodegradable and bioerrodible. The resorbable polymer or combination of polymers can include any polymer or combination of polymers that are miscible in a mild organic polymer solvent, for example, acetonitrile. The mild organic polymer solvent is also capable of solubulizing all of the antibiotics used to make the coating, for example, a rifamycin class of antibiotic (e.g. rifampin) and a second antibiotic selected from the group consisting of tetracyclines, penicillin, ampicillin, cefazolin, clindamycin, erythromycin, levofloxacin, or vancomycin.

The resorbable polymer can include homopolymers, copolymers, block copolymers and combinations thereof. The resorbable polymers can include one or more members of the group of cyclic esters, for example, lactones, including butyrolactone, valerolactone, caprolactone, propiolactone; dioxanones; glycolide; lactide and combinations thereof. In some embodiments, suitable cyclic esters may possess a heteroatom, such as oxygen, adjacent to the a-carbon. Suitable cyclic esters used as monomers can include glycolide, L(−)-lactide, D(+)-lactide, meso-lactide, p-dioxanone, 1,4-dioxan-tone, 1,5-dioxepan-2-one, epsilon (ε)-caprolactone, delta (δ) valerolactone, gamma (γ)-butyrolactone, beta-propiolactone, and combinations thereof. In some embodiments, the resorbable polymer can be a member of a biocompatible dicarboxylic acid and polyester such as aliphatic and aromatic polyarylates.

Resorbable polymers contemplated as useful, and derived from the above cyclic esters, can include polylactic acid, polyglycolic acid and copolymers and mixtures thereof such as poly(L-lactic acid) (PLLA), poly(D,L-lactide), poly(lactic acid-co-glycolic acid), 50/50 (DL-lactide-co-glycolide), polydioxanone, polycaprolactone and co-polymers and mixtures thereof such as poly(D,L-lactide-co-caprolactone). In a preferred embodiment, the resorbable polymer is poly(D,L-lactide-co-ε-caprolactone), such as is commercially available as Purasorb PLC 7015 70/30(%) L-lactide/ε-caprolactone copolymer (1.5 g/dL) from PURAC America (Lincolnshire, Ill., USA). When the resorbable polymer is poly-L-lactide-ε-co-caprolactone, the mol % of the caprolactone content in the poly-L-lactide-ε-co-caprolactone copolymer may range from about 15 mol % to about 40 mol %. The ratio of lactide monomer to caprolactone monomer in the poly-L-lactide-ε-co-caprolactone polymer may range from about 50:50 to about 85:15, more preferably from about 65:35 to about 75:25. The resorbable polymers are preferably compatible and miscible with organic solvents that are not overtly toxic, for example, acetonitrile. The molecular weight of the resorbable polymers can vary from about 10,000 Da. to about 200,000 Da. For example, the resorbable polymer has a molecular weight ranging from about 1,000 Da. to about 200,000 Da. or from about 50,000 Da. to about 200,000 Da. or from about 100,000 Da. to about 200,000 Da. or from about 1,000 Da. to about 150,000 Da. or from about 1,000 Da. to about 100,000 Da. or from about 1,000 Da. to about 75,000 Da. or from about 1,000 Da. to about 50,000 Da. or from about 1,000 Da. to about 25,000 Da.

In orthopedic surgical procedures, target organisms are those microbial organisms which become associated with a device or may have access to internal tissues such as blood, muscle, cartilage, and bone inside the subject and cause an infection. Thus, any organism that has the potential to enter surreptitiously and colonize at a surgical site or area of orthopedic repair and trauma may be targeted in accordance with the present technology. Particularly relevant target organisms are micro-organisms such as Gram-positive and Gram-negative bacteria along with yeasts. For example, microbial pathogens may become associated with devices such as bone fixator rods and nails and cause sepsis and osteomyelitis. In addition, bacterial infections may spread to other internal tissues and organs, including the heart. Microorganisms such as bacteria and yeasts can bind to proteins involved in thrombus formation on the surface of the medical device, such as fibrin/fibrinogen and fibronectin. The interaction can be mediated by the production of a number of microbial surface components recognizing adhesive matrix molecules. In some bacterial infections, for example, Staphylococcus aureus, these include the fibrinogen-binding clumping factors A and B and the fibronectin-binding protein (FnbA). Other microorganisms involved in medical device-related infections can include Staphylococcus epidermidi, Streptococcus ssp., and Gram negative bacilli (Clin Infect Dis 2003; 36(9):1157-1161; J Bone Join Surg Am 1996; 78(4):512-23). Importantly, disruption of the skin barrier as a result of bodily trauma, for example, accidents, military casualties and the like, may allow the entry of Gram-negative bacteria, including Klebsiella, Enterobacter, Acinetobacter, Pseudomonas and Escherichia.

In particular, organisms that colonize the skin of the subject are targeted, since these organisms may enter the subject at the site where the orthopedic implant was inserted. These opportunistic bacteria become associated with the implant leading to masking from the immune system and cause an infection. Particularly relevant target pathogens are Gram-positive bacteria, in particular Staphylococcus and Enterococcus species. The viridans group streptococci and Streptococcus bovis are also targets implicated in infective endocarditis associated with valves, such as heart valves. A particular target is Staphylococcus aureus, as represented by strain NCTC 8325 and methicillin resistant strains which presently cause significant problems in hospital environments. Further targets are Staphylococcus epidermidis, represented by strain NCTC 11047, Coryneforms and Diptheroids, for example, Corynebacteria diptheriae represented by strain NCTC 5002 and C. xerosis represented by strain ATTC 7711, and yeasts such as Candida albicans, represented by strain ATCC 26555. Some of these bacteria are known to produce fibronectin binding surface proteins and are capable of adhering to orthopedic implants and related devices.

The present antibacterial coatings for orthopedic implants include broad-spectrum antimicrobial agents to combat a number of pathogens provided there is little or no toxicity or allergy for the subject. Suitable antimicrobial agents may have at least one or more of the following properties: (1) the ability to prevent growth and/or replication and/or to kill pathogens which become associated with the orthopedic implant through their ability to bind to blood, muscle and osseous tissue; (2) the agents should be non-toxic to the subject and without adverse side effects; (3) the agents should be non-allergenic to the subject; (4) the agents should act locally, i.e. at the site of trauma or surgical bed and not eliminate the natural flora of the subject; (5) the agents should be miscible and remain solublized in the organic solvents used to disperse the resorbable polymer; (6) the agents should be stable in the resorbable polymer and also in the coating when applied to the orthopedic implant, and when the orthopedic implant is utilized in vivo; (7) the agents should preferably be cheap and readily available/easy to manufacture; and (8) the agents should be sufficiently potent that pathogen resistance does not develop (to any appreciable degree). Preferably, a combination of suitable antimicrobial agents is utilized, including a rifamycin agent and a second antibiotic selected from the group consisting of tetracyclines, penicillin, ampicillin, cefazolin, clindamycin, erythromycin, levofloxacin, or vancomycin.

Tetracycline antibiotics refer to a number of antibiotics of either natural, or semi-synthetic origin, derived from a system of four linearly annealed six-membered rings (1,4,4a,5,5a,6,11,12a-octahydronaphthacene) with a characteristic arrangement of double bonds. The tetracycline antibiotic can include one or more tetracyclines, and/or semi-synthetic tetracyclines such as doxycycline, oxytetracycline, demeclocycline, lymecycline, chlortetracycline, tigecycline and minocycline. A preferred tetracycline is minocycline or minocycline hydrochloride. Minocycline is a semisynthetic derivative of tetracycline and has a IUPAC designation of 4,7-Bis(dimethylamino)-1,4,4a,5,5a,6,11,12a octahydro-3,10,12,12a-tetrahydroxy-1,1′-dioxo-2naphthacenecarboxamide monohydrochloride. Minocycline is commercially available as Dynacin, Minocin, Minocin PAC, Myrac, Solodyn, Sumycin, Terramycin, Tetracyn, and Panmycin from various suppliers. The amount of tetracycline present in the resorbable antimicrobial coating can range from about 5 μg/cm² to about 1000 μg/cm², or from about 10 μg/cm² to about 800 μg/cm²

Rifamycin class of antibiotics is a subclass of antibiotics from the Ansamycin family of antibiotics. The present antibiotic agent or agents can include one or more Rifamycin antibiotics from the group rifamycin B, rifampin or rifampicin, rifabutin, rifapentine and rifaximin. Rifampin is commercially available as Rifadin and Rimactane from Sanofi-Aventis U.S. LLC. (Bridgewater, N.J., USA).

Rifampin is the most powerful anti-staphylococcal agent approved for human use (J Bone Joint Surg 2001; 83A:1878-1890; Rev Infectious Diseases 1983; 5(3):S412-S417). In a coating, a powerful agent is of advantage because of inherent limitations of the amount of antibiotic agent that can be carried in the polymer matrix or limitations to the thickness of the coating which in turns limits the amount of antibiotic delivered per unit surface area. Since the vast majority of orthopedic device-related infections are due to staphylococci, rifampin is a primary choice for antibiotic in an anti-infective coating. However, bacterial resistance to rifampin is easily generated by a single point mutation of DNA-directed RNA polymerase. For this reason, rifampin is never administered alone but is combined with another agent that would act against any rifampin-resistant micro-organisms that would arise. See, Mader et al., Antibiotic Therapy for Musculoskeletal Infections, J Bone Joint Surg 2001; 83A:1878-1890.

Rifampin has been used in combination with minocycline in local anti-infective technologies in devices such as catheters and surgical meshes. In this invention, a range of other antibiotics in addition to minocycline with broad spectrum of activity including antistaphylococcal activity have been identified as useful in combination with resorbable polymer coating technology.

A particularly preferred combination of antibiotic agents includes minocycline and rifampin. Both minocycline and rifampin are excellent anti-staphyloccocal agents, and are in fact active against MRSA and MRSE. Because minocycline and rifampin are both more lipophilic than other antibiotics, high concentrations of these antibiotics in organic solvents can be generated. The amount of minocycline and rifampin in the resorbable polymer mixture ranges from about 0.1 mg/mL to about 100 mg/mL, or from about 1 mg/mL to about 90 mg/mL, or from about 1 mg/mL to about 80 mg/mL, or from about 1 mg/mL to about 70 mg/mL, or from about 1 mg/mL to about 50 mg/mL or from about 1 mg/mL to about 30 mg/mL, or from about 10 mg/mL to about 20 mg/mL, or from about 5 mg/mL to about 100 mg/mL, or from about 25 mg/mL to about 100 mg/mL, or from about 50 mg/mL to about 100 mg/mL or from about 75 mg/mL to about 100 mg/mL. In some embodiments, the amount of antimicrobial in the mixture is from about 5 mg/mL to about 15 mg/mL.

### Methods of Making the Antibiotic Coated Orthopedic Implant

The present technology also relates to a method for making an antibiotic coated orthopedic implant. The method includes: a) dissolving a resorbable polymer in a suitable organic polymer solvent forming a resorbable polymer mixture; b) adding rifampin and a second selected antibiotic to an organic solvent forming an antibiotic solution; c) mixing the resorbable polymer mixture with the antibiotic solution forming a coating solution; d) applying the coating solution to the one or more surfaces of the metal implant forming an adhesive coating layer; and e) evaporating the organic solvent from the adhesive coating layer to form an antibiotic containing resorbable polymer matrix coated orthopedic implant.

The resorbable polymer mixture includes any type of polymer mixture comprising a polymer such as a resin, solution, dispersion and the like and mixing the resorbable polymer in a quantity of compatible organic polymer solvent. The resorbable polymer can either be powdered, particulate (granules, chips, particles and the like) or liquid and can be solubulized or miscible in a compatible organic solvent.

Generally, the organic polymer solvent is an organic solvent that will keep the antibiotic agents and polymer in solution (after mixture with the antibiotic and organic antibiotic solvent, prior to evaporating, in the processes of the present technology), and prevent their precipitation. Furthermore, the organic polymer solvent can solubulize or be miscible with one or more resorbable polymers. In addition, the organic polymer solvent is not overtly toxic, i.e., is not a known carcinogen or severe irritant, with sufficient volatility to allow air drying of the coating at room temperature within seconds to hours. Preferred organic polymer solvents include acetonitrile, tetrahydrofuran, dimethylsulfoxide (DMSO), and mixtures thereof. The resorbable polymer mixture can be prepared by mixing an amount of resorbable polymer with an organic polymer solvent. The final concentration of the resorbable polymer can range from about 1% to about 25% by weight of the final resorbable polymer mixture.

The resorbable polymer mixture can then be mixed with a solution containing the antibiotic agents. A solid and/or liquid form of the antibiotic agents can be mixed with an organic antibiotic solvent that will allow the antibiotic solution produced thereby to be easily mixed with the resorbable polymer mixture containing a compatible organic polymer solvent. In some embodiments, the antibiotic solution can be made by adding an amount of each antibiotic with an organic solvent that is miscible with the solvent used to dissolve the polymer and must evaporate readily after the coating solution is applied to the metal surface.

The FDA has classified organic solvents as class 1, 2, or 3 based upon toxicity. Class 1 solvents include benzene, carbon tetrachloride 1,2-dichloroethane, 1,1-dichloroethene, and 1,1,1-trichloriethane. These solvents are either carcinogenic, highly toxic, or environmental hazards and should not be utilized in the manufacture of either devices or drugs. Class 2 solvents are associated with less severe toxicity effects and can be used providing residual levels are minimal. Class 3 solvents are of low toxic potential to humans. Details regarding these solvent classes can be found in FDA guidance document Q3C Impurities: Residual Solvents.

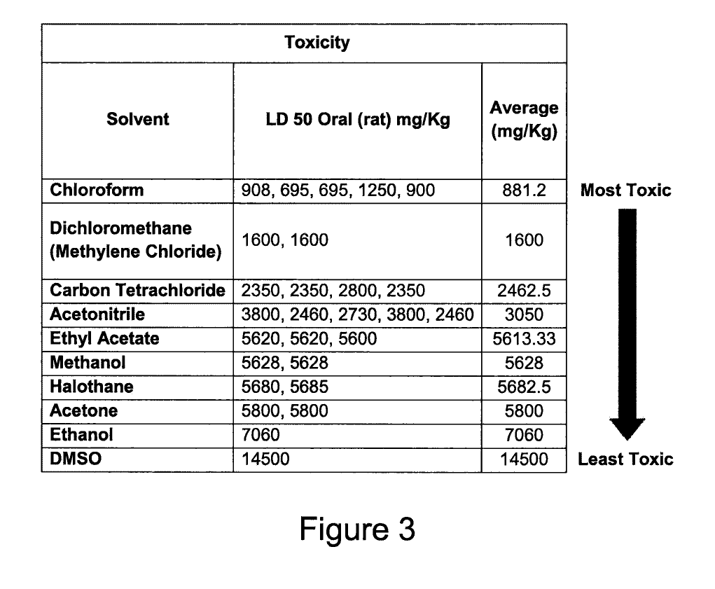

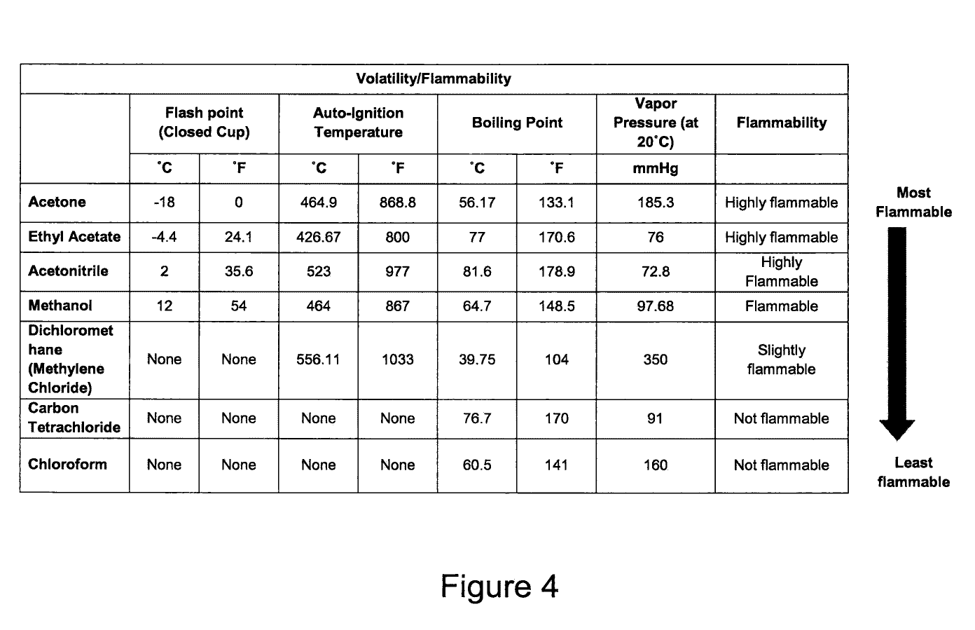

In considering suitable solvents, certain class 2 solvents are too hazardous for use in a manufacturing production operation. These include chloroform and dichloromethane, which are highly toxic (see FIG. 3). Solvents that are undesirable for use in a production operation due to high flammability include acetone and ethyl acetate (see FIG. 4).

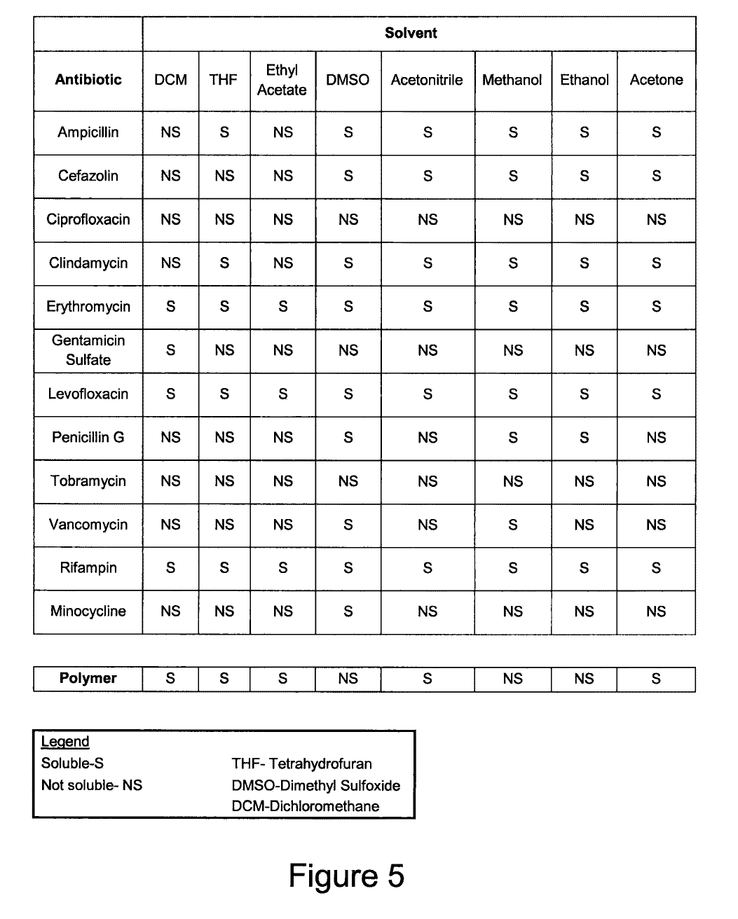

Eight common solvents that are either class 2 or class 3 may be considered as potential solvents for the antibiotic component: tetrahydrofuran, dimethylsulfoxide (DMSO), acetonitrile, methanol, and ethanol. Potentially useful antibiotic included tetracyclines, rifampin, penicillin, ampicillin, cefazolin, ciprofloxacin, clindamycin, gentamicin sulfate, penicillin, tobramycin, and vancomycin. It has been found that DMSO, acetonitrile, methanol, and ethanol, which are class 2 or class 3 common solvents, are suitable solvents for most of the antibiotics, except for ciprofloxacin, gentamicin sulfate, and tobramycin (See FIG. 5). Solvents that do not form solutions of most of the antibiotics are ethyl acetate, dichloromethane, and tetrahydrofuran, with the exception that these three solvents dissolved erythromycin and levofloxacin.

The resorbable polymers of preference, lactide-co-caprolactone, are soluble in dichloromethane, tetrahydrofuran, ethyl acetate, acetonitrile, dimethyl sulfoxide, and acetone. Because of flammability or toxicity concerns, only acetonitrile and tetrahydrofuran are considered suitable for a manufacturing coating operation.

Taken together, antibiotics that are soluble in acceptable class 2 or 3 organic solvents (DMSO, acetonitrile, methanol, ethanol) that are known to be compatible with the acetonitrile or THF polymer solutions included penicillin, ampicillin, cefazolin, clindamycin, erythromycin, levofloxacin, vancomycin, minocycline, and rifampin.

In some embodiments, the antibiotic solvent is the same as the polymer solvent; in other embodiments the antibiotic solvent is different than the polymer solvent. In particular, the antibiotic solvent may be an organic solvent as described above, or a polar solvent such as small carbon chain alcohol, for example, 2-propanol (isopropanol), n-propanol, ethanol or methanol. The concentration of each antibiotic, rifampin and a second antibiotic selected from the group consisting of tetracyclines, penicillin, ampicillin, cefazolin, clindamycin, erythromycin, levofloxacin, or vancomycin, in the resorbable polymer mixture can range from about 0.1 mg/mL to about 100 mg/mL, preferably from about 1 mg/mL to about 30 mg/mL. The amount (density of coverage) of each antibiotic coated on the orthopedic implant can range from about 10 μg/cm² to about 1000 μg/cm², or preferably, from about 50 μg/cm² to about 200 μg/cm². In various embodiments, the amount ranges from about 10 μg/cm² to about 175 μg/cm², or from about 10 μg/cm² to about 150 μg/cm², or from about 10 μg/cm² to about 100 μg/cm², or from about 10 μg/cm² to about 75 mg/cm², or from about 20 μg/cm² to about 200 μg/cm² or from about 50 μg/cm² to about 200 μg/cm², or from about 75 μg/cm² to about 200 μg/cm² or from about 100 μg/cm² to about 200 μg/cm², or from about 150 μg/cm² to about 200 μg/cm².

Once the antibiotic solution comprising the two antibiotic agents has been mixed with the resorbable polymer mixture, the resulting coating solution can be applied to at least one surface of the orthopedic implant. The application of the coating solution to the orthopedic implant can be accomplished in any appropriate manner, including application methods known to those in the art of coated medical devices. For example, a coating solution having a 5%-10% resorbable polymer mixture (5-10% by weight of the resorbable polymer in acetonitrile) containing poly-L-lactide-ε-co-caprolactone (viscosity 1.2-1.8 g/dL) and 5-30 mg/mL of each antibiotic (final concentration of each antibiotic in the coating solution) can be brush coated, spray coated, roll coated, printed, sputtered, and dip coated. A preferred method for applying the coating solution to an orthopedic implant surface is spray coating with ultrasonic atomization assisted spraying. The coating solution is injected through a spraying applicator as a thin film until it flows on a vibrating surface (frequency>20 kHz) which then breaks the liquid film into fine droplets. This procedure is known as ultrasonic atomization. The droplet size, in the case of ultrasonic atomization, depends upon the ultrasonic parameters such as frequency and intensity, operating parameters such as liquid flow rate and physiochemical properties such as density, viscosity, surface tension and vapor pressure of the coating solution. The flow rate can be adjusted to 1-100 mL per minute.

As noted above, the orthopedic implant surface to be coated with the coating solution of the present technology is preferably metallic and can be textured. Thin layers of coating solution can be applied to the orthopedic implant surface, each layer ranging from 1 micron to about 50 microns in width, thereby forming an adhesive coating layer. In some embodiments, the coating solution can be applied as several coats, ranging from 1 to about 100 depending on the desired thickness of the final adhesive coating layer, the type of orthopedic implant being implanted, the amount of antibiotic needed to be eluted from the adhesive coating layer and the site of the implantation. The final thickness of the adhesive coating layer disposed on a surface of an orthopedic implant can range from about 1 μm to about 200 μm, or preferably, from about 10 μm to about 50 p.m. In various embodiments, the thickness ranges from about 1 μm to about 150 μm, or from about 1 μm to about 100 μm, or from about 1 μm to about 75 μm, or from about 1 μm to about 50 μm, or from about 1 μm to about 25 μm, or from about 10 μm to about 200 μm, or from about 50 μm to about 200 μm or from about 100 μm to about 200 μm, or from about 125 μm to about 200 μm or from about 150 μm to about 200 μm, or from about 175 μm to about 200 μm.

Once the adhesive coating layer has been applied to a surface of an orthopedic implant, the orthopedic implant can be dried to remove or evaporate the organic solvent. While several possible approaches can be undertaken to evaporate the residual organic solvent, a preferred method of evaporation involves leaving the wet orthopedic implant at room temperature (20-22° C.) for a period of 1 minute to 48 hours. Other approaches can include placing the wet orthopedic implant in an oven set to a temperature ranging from about 0° C. to 50° C., more preferably set to about 10° C. to about 30° C. for a period of 1 minute to 48 hours. In an alternate approach for evaporating the organic solvent, the wet orthopedic implant can be placed in an oven set to a temperature ranging from about 10° C. to about 50° C. with an environment of compressed gas, for example, argon, nitrogen, helium or air.

Optional post coating and drying steps can further include sterilization of the antibiotic coated orthopedic implant. For gamma (γ) irradiation, the coated orthopedic implant is irradiated at a dose of about 1.5-4 Mrad Irradiation of the resorbable antibiotic coated orthopedic implant can be accomplished in an atmospheric, inert atmosphere or vacuum. For example, the coated orthopedic implant may be packaged in an oxygen impermeable package during the irradiation step. Inert gases, such as nitrogen, argon, and helium may also be used. When vacuum is used, the packaged material may be subjected to one or more cycles of flushing with an inert gas and applying the vacuum to eliminate oxygen from the package. Examples of package materials include metal foil pouches such as aluminum or Mylar® coating packaging foil, which are available commercially for heat sealed vacuum packaging. Irradiating the coated orthopedic implant in an inert atmosphere reduces the effect of oxidation and the accompanying chain scission reactions that can occur during irradiation. Oxidation caused by oxygen present in the atmosphere present in the irradiation is generally limited to the surface of the polymeric material. In general, low levels of surface oxidation can be tolerated. Irradiation such as gamma-irradiation can be carried out on the coated orthopedic implant at specialized installations possessing suitable irradiation equipment.

The materials and processes of the present technology are illustrated in the following non-limiting examples.

### EXAMPLES

### Example 1

### Method for Making an Intramedullary Nail Coated With Minocycline and Rifampin

A polymer resin comprising poly-L-lactide-co-ε-caprolactone (70:30) blend (Purasorb PLC 7015 70/30(%) L-lactide/ε-caprolactone copolymer (1.5 g/dL) from PURAC America (Lincolnshire, Ill., USA) is dissolved in acetonitrile forming a resorbable polymer mixture. The resorbable polymer mixture is mixed with a methanol solution containing minocycline and rifampin. The combined polymer and antibiotic solution forms a coating solution that was applied to a metal cylinder. The final amount of the poly-L-lactide-co-ε-caprolactone used to coat each cylinder is 5% by weight. The initial concentration of the antibiotics in methanol is 50 mg/ml of methanol; after addition of 25 mL of antibiotic solution to 75 ml of the acetonitrile polmyer solution, the final antibiotic concentration in the methanol-acetonitrile-polymer solution is 12.5 mg/ml. The cylinder is dip coated with the coating solution and air dried to evaporate the acetonitrile. To measure the release profile of the two antibiotics from the resorbable polymer matrix coated cylinder, the coated cylinder is soaked in phosphate buffered saline at 37° C. for a period ranging from 0 to 10 days.

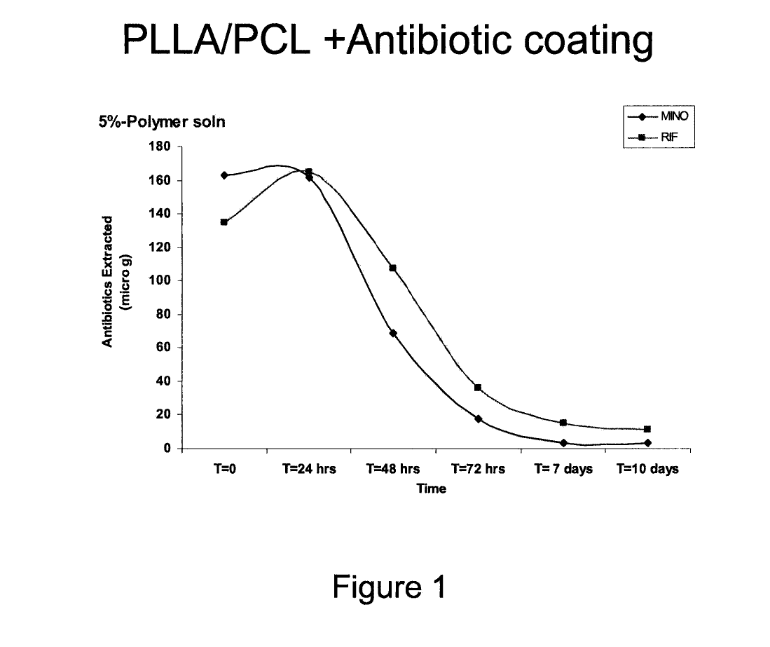

The elution profile of the minocycline and rifampin is shown in FIG. 1. The elution particle is measured from a coated cylinder immersed in phosphate buffered saline for 0 to 10 days at 37° C. The amount of each antibiotic released is determined using high performance liquid chromatography (HPLC) in accordance with the teachings of the present technology.

As can be seen from FIG. 1, both antibiotics eluted from the coated cylinder roughly in an equal measure. From the observed elution profile, it can be observed that approximately 80% of the antibiotics had eluted over a period approximating four days. Such an antibiotic elution profile may be considered prophylactically suitable to ensure a high dose of each antibiotic agent for the prevention of bacterial infection.

### Example 2

### Elution Profile of an Antibiotic Coated Intramedullary Nail

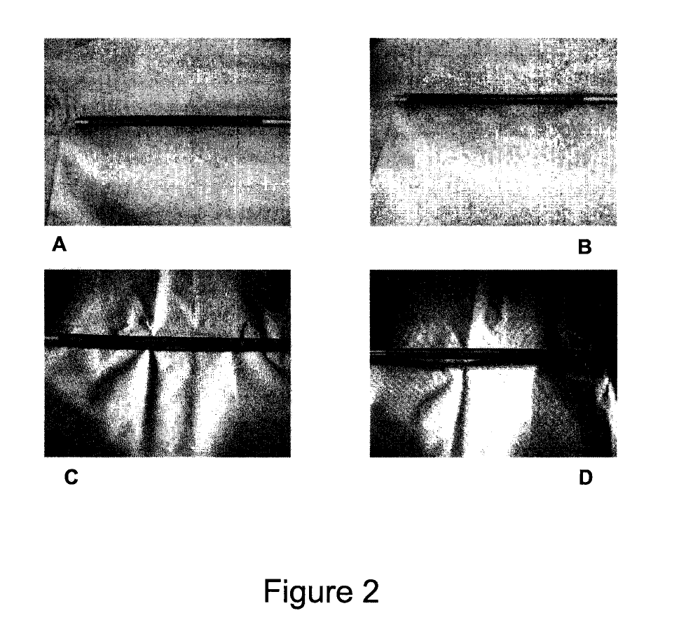

An 11 mm diameter intramedullary nail (Uniflex® Femoral Nail System, 14 mm proximal nail diameter, 3 mm proximal wall thickness, 2 mm distal wall thickness and 32 cm in length, Biomet Trauma Biomet Spine (Parsippany, N.J., USA) is coated with a coating solution forming a 20 μm adhesive coating layer using an ultrasonic assisted spraying method. The resorbable polymer matrix coated intramedullary nail is inserted into a cadaver femur and was immediately removed to visually assess the amount of antibiotic coating removed during the implantation and extraction processes. The intramedullary canal was prepared prior to insertion of the nail with a reamer of a size appropriate to the diameter of the nail. After removing the femoral nail from the cadaver, photographs are taken of the recovered orthopedic implant. As shown in FIG. 2, the majority of the adhesive coating layer still remains adhered to the femoral nail as the color of the regions still retaining the adhesive coating layer is readily quantifiable. Such methods for preparing orthopedic implants described herein readily produce antibiotic coated implant devices that withstand the abrasion and stripping rigors of orthopedic surgeries. The operative procedures used to place and remove the orthopedic implant are often performed with metal instruments and the like that tend to wear off superficially coated non-adhesive coating layers. In contrast, the present methods provide for antibiotic coated orthopedic implants that have fairly resistant adhesive antibiotic coatings that can withstand the rigors of orthopedic surgeries as shown by the substantial retention of the adhesive coating layer on the implanted intramedullary nail shown in FIG. 2. Given that greater than about 70% of the adhesive coating still remains attached on the intramedullary nail of FIG. 2, it is believed that sufficient amounts of the two antibiotic agents elute from the coating (as shown in FIG. 1) to provide concentrations of each antibiotic above the minimum inhibitory concentration (MIC) for all of the common bacterial pathogens likely to cause a postoperative infection at the site of implantation.

The embodiments and the examples described herein are exemplary and not intended to be limiting in describing the full scope of compositions and methods of the present technology. Equivalent changes, modifications and variations of embodiments, materials, compositions and methods can be made within the scope of the present technology, with substantially similar results.

### Non-limiting Discussion of Terminology:

The headings (such as “Introduction” and “Summary”) and sub-headings used herein are intended only for general organization of topics within the present technology, and are not intended to limit the disclosure of the present technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The citation of references herein does not constitute an admission that those references are prior art or have any relevance to the patentability of the technology disclosed herein. Any discussion of the content of references cited in the Introduction is intended merely to provide a general summary of assertions made by the authors of the references, and does not constitute an admission as to the accuracy of the content of such references. All references cited in the “Description” section of this specification are hereby incorporated by reference in their entirety.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of” Thus, for any given embodiment reciting ingredients, components or process steps, Applicants specifically envision embodiments consisting of, or consisting essentially of, such ingredients, components or processes excluding additional ingredients, components or processes (for consisting of) and excluding additional ingredients, components or processes affecting the novel properties of the embodiment (for consisting essentially of), even though such additional ingredients, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

What is claimed is:

1. An orthopedic implant comprising:
a metal substrate;
a coating on a surface of the substrate comprising a resorbable polymer impregnated with an admixture of a rifamycin antibiotic and a second antibiotic selected from the group consisting of tetracyclines, penicillin, ampicillin, cefazolin, clindamycin, erythromycin, levofloxacin, vancomycin, and mixtures thereof.

2. The orthopedic implant of claim 1, wherein the metal substrate comprises a hip implant, a knee implant, an elbow implant, a prosthetic frame, bone plate, a bone prosthesis, a small joint prosthesis, a rod, a pin, a hook, a nail, a bone screw, a spacer or a cage.

3. The orthopedic implant of claim 1, wherein the metal substrate comprises gold, silver, stainless steel, platinum, palladium, iridium, iron, nickel, copper, titanium, aluminum, chromium, cobalt, molybdenum, vanadium, tantalum or alloys thereof.

4. The orthopedic implant of claim 1, wherein the coating has a thickness of less than 200 μm.

5. The orthopedic implant of claim 1, wherein the resorbable polymer includes one or more cyclic esters, selected from the group consisting of butyrolactone, valerolactone, caprolactone, propiolactone, dioxanones, glycolide, lactide and combinations thereof.

6. The orthopedic implant of claim 5, wherein the resorbable polymer is selected from the group consisting of polylactic acid, polyglycolic acid and copolymers and mixtures thereof.

7. The orthopedic implant of claim 5, wherein the resorbable polymer has a molecular weight ranging from about 10,000 Da. to about 200,000 Da.

8. The orthopedic implant of claim 7, wherein the resorbable polymer comprises poly(D,L-lactide-co-ε-caprolactone).

9. The orthopedic implant of claim 1, wherein the concentration of each of rifampin and the second antibiotic coated on the metal substrate's one or more surfaces ranges from 10 μg/cm² to about 1000 μg/cm².

10. The orthopedic implant of claim 1, wherein the second antibiotic comprises minocycline.

11. An orthopedic implant for implantation in contact with a bone or soft tissue of a subject, the implant comprising:
a metal substrate;
a coating on a surface of the substrate, comprising a resorbable polymer impregnated with an admixture of a rifamycin antibiotic and a second antibiotic selected from the group consisting of tetracyclines, penicillin, ampicillin, cefazolin, clindamycin, erythromycin, levofloxacin, vancomycin, and mixtures thereof, wherein the coating is operable to release the rifamycin and second antibiotics in an antimicrobially effective amount at the site of the implantation.

12. The orthopedic implant of claim 11, wherein the coating has a thickness of less than 200 μm.

13. The orthopedic implant of claim 11, wherein the resorbable polymer is selected from the group consisting of polylactic acid, polyglycolic acid and copolymers and mixtures thereof, and has a molecular weight ranging from about 10,000 Da. to about 200,000 Da.

14. The orthopedic implant of claim 11, wherein the concentration of each of rifampin and the second antibiotic coated on the metal substrate's one or more surfaces ranges from 10 μg/cm² to about 1000 μg/cm².

15. The orthopedic implant of claim 11, wherein the second antibiotic comprises minocycline.

16. A method for making an orthopedic implant, the method comprising:
a) dissolving a resorbable polymer in a suitable Class 2 or Class 3 organic polymer solvent forming a resorbable polymer mixture;
b) adding rifampin and a second antibiotic selected from the group consisting of tetracyclines, penicillin, ampicillin, cefazolin, clindamycin, erythromycin, levofloxacin, vancomycin, and mixtures to a Class 2 or Class 3 organic antibiotic solvent forming an antibiotic solution;
c) mixing the resorbable polymer mixture with the antibiotic solution forming a coating solution;
d) applying the coating solution to a surface of a metal implant substrate forming an coating layer; and
e) evaporating the solvents from the coating layer to form an antibiotic containing resorbable polymer matrix coated orthopedic implant.

17. The method of claim 16, wherein the organic polymer solvent is selected from the group consisting of acetonitrile, tetrahydrofuran, dimethylsulfoxide, and mixtures thereof.

18. The method of claim 16, wherein the organic antibiotic solvent is selected from the group consisting of dimethylsulfoxide, acetonitrile, methanol, 2-propanol, n-propanol, ethanol, and mixtures thereof.

19. The method of claim 16, wherein the resorbable polymer is selected from the group consisting of polylactic acid, polyglycolic acid and copolymers and mixtures thereof, and has a molecular weight ranging from about 10,000 Da. to about 200,000 Da.

20. The method of claim 16, wherein the concentration of each of rifampin and the second antibiotic coated on the metal substrate's one or more surfaces ranges from 10 μg/cm² to about 1000 μg/cm².

21. The method of claim 16, wherein the second antibiotic comprises minocycline.

22. The method of claim 16, wherein the coating has a thickness of less than 200 μm.
